(19) United States
(12) Patent Application Publication
(10) Pub. No.: US 2015/0335781 A1
(43) Pub. Date: Nov. 26, 2015

(54) BONE PROSTHESIS MATERIAL AND METHOD FOR MANUFACTURING SAME

(71) Applicant: OLYMPUS TERUMO BIOMATERIALS CORP., Tokyo (JP)

(72) Inventors: Kazuaki HASHIMOTO, Chiba (JP); Hikaru INOUE, Tokyo (JP)

(73) Assignee: OLYMPUS TERUMO BIOMATERIALS CORP., Tokyo (JP)

(21) Appl. No.: 14/719,661

(22) Filed: May 22, 2015

Related U.S. Application Data
(63) Continuation of application No. PCT/JP2013/082012, filed on Nov. 28, 2013.
(60) Provisional application No. 61/730,757, filed on Nov. 28, 2012.

(51) Int. Cl. A61L 27/04; A61L 27/42; A61L 27/12

(52) U.S. Cl. CPC A61L 27/047; A61L 27/12; A61L 27/425; A61L 2430/02

(57) ABSTRACT

A bone prosthesis material that promotes calcium ion elution while retaining its effect on osteoclasts so that its binding to a bone can be achieved at an early stage. The bone prosthesis material that is composed of a plurality of β-tricalcium phosphate (β-TCP) phases containing monovalent cations in solid solution at different concentrations. Being a composite phase of β-TCP phases containing monovalent cations in solid solution at different concentrations, the bone prosthesis material promotes calcium ion elution while retaining its effect on osteoclasts, and thus enables its binding to a bone at an early stage.

### CROSS-REFERENCE TO RELATED APPLICATIONS

This application is a continuation of International Application PCT/JP2013/082012 filed on Nov. 28, 2013, which claims benefit of U.S. Provisional Application No. 61/730,757 filed on Nov. 28, 2012. The entire contents of these applications are hereby incorporated by reference.

### TECHNICAL FIELD

The present invention relates to a bone prosthesis material and a method for manufacturing the same.

### BACKGROUND ART

Bone prosthesis materials are used as substitute materials for defective or lost bones due to accidents and diseases. An important function of a bone prosthesis material lies in how fast the bone prosthesis material binds to the surrounding bone tissue. Binding of a bone prosthesis material to bone tissue requires two steps: (1) calcium ions eluted from the surface of the bone prosthesis material allow osteoclasts to migrate in the vicinity of the bone prosthesis material; and (2) the osteoclasts adhere to the surface of the bone prosthesis material and dissolve the bone prosthesis material to thereby operate the bone metabolism cycle. That is, to enhance the function of the bone prosthesis material, the balance between the bone formation rate and the absorption rate of β-TCP is required to be controlled.

β-Tricalcium phosphate (β-TCP), which is widely used as a bone prosthesis material, has solubility high enough to allow osteoclasts to migrate. A material composed of β-TCP has a highly soluble surface, to which osteoclasts have difficulty in adhering. This causes an insufficient increase in the activity of the osteoclasts. Accordingly, an effort has been made to reduce the solubility by substitution and solid solubilization using cations such as sodium ions in β-TCP (for example, see Patent Literature 1.).

### CITATION LIST

### Patent Literature

### {PTL 1}

Japanese Unexamined Patent Application, Publication No. 2001-259016

### Non Patent Literature

### {NPL 1}

M. Yashima, A. Sakai, T. Kamiyama, A. Hoshikawa, “Crystal structure analysis of β-tricalcium phosphate Ca₃(PO₄)₂ by neutron powder diffraction”, J. Solid State Chem., 175, 272-277 (2003).

### {NPL 2}

Yoshida Katsumi, Kondo Naoki, Kita Hideki, Mitamura Masanori, Hashimoto Kazuaki, Toda Yoshitomo, “Effect of Substitutional Monovalent and Divalent Metal Ions on Mechanical Properties of β-Tricalcium Phosphate”, Journal of the American Ceramic Society, Vol. 88 (8), pp. 2315-2318 (2005).

### {NPL 3}

Katsumi YOSHIDA, Yoshinari FUKUHARA, Kazuaki HASHIMOTO, Yoshitomo TODA, Masamitsu IMAI and Toyohiko YANG, “Sinterability and Mechanical Properties of β-Tricalcium Phosphates Doped with Both Na and Mg Ions” Journal of the Society of Inorganic Materials, Japan, (Vol. 16) No. 340, p. 165-170 (2009).

### {NPL 4}

Kazuaki Hashimoto, Naoyuki Matsumoto, and Hirofumi Shibata, “Various Metal Ions-substituted Tricalcium Phosphate-based Biomaterials”, Zairyo No Kagaku To Kogaku, Vol. 49, No. 6, pp. 250-255 (2012).

### {NPL 5}

Naoyuki Matsumoto, Katsumi Yoshida, Kazuaki Hashimoto, Yoshitomo Toda, Dissolution mechanisms of β-tricalcium phosphate doped with monovalent metal ions, Journal of the Ceramic Society of Japan, Vol. 118, No. 1378 (June), P 451-457 (2010).

### SUMMARY OF INVENTION

### Technical Problem

Use of the bone prosthesis material according to Patent Literature 1 has successfully resulted in an increase in the activity of the osteoclasts on the material, whereas it has been found that the initial amount of calcium ions eluted was lacking and insufficient to migrate the osteoclasts in a living body.

An object of the present invention is to provide a bone prosthesis material that can bind to a bone at an early stage by promoting elution of calcium ion while retaining its effect on osteoclasts and a method for manufacturing the same.

### Solution to Problem

A first embodiment of the present invention provides a bone prosthesis material that is composed of a plurality of β-tricalcium phosphate (β-TCP) phases containing monovalent cations in solid solution at different concentrations.

In the bone prosthesis material according to the first embodiment, the plurality of β-TCP phases containing monovalent cations in solid solution at different concentrations may form a heterogeneously mixed phase.

The bone prosthesis material according to the first embodiment may include a β-TCP phase containing monovalent cations in solid solution and a non-solid-solution β-TCP phase.

In the first embodiment, the monovalent cations may be sodium ions.

The bone prosthesis material according to the first embodiment may be formed by mixing particles of β-TCP containing monovalent cations in solid solution at different concentrations, shaping the mixture by a pressing method or a cutting shaping method into a shape suitable for a bone prosthesis material, that is, a cylinder, a rectangular parallelepiped, a polygonal column, a sphere, an ellipsoid, a granule, or a shape formed using three-dimensional data so as to precisely fit to a bone defect, and subsequently firing the shaped product under conditions of 1100° C. or more and 1200° C. or less for 5 minutes or more and 60 minutes or less.

A second embodiment of the present invention provides a method for manufacturing a bone prosthesis material comprising steps of: mixing particles of β-TCP containing monovalent cations in solid solution and particles of non-solid-solution β-TCP to provide a mixture; and shaping the mixture by a pressing method or a cutting shaping method into a shape suitable for a bone prosthesis material, that is, a cylinder, a rectangular parallelepiped, a polygonal column, a sphere, an ellipsoid, a granule, or a shape formed using three-dimensional data so as to precisely fit to a bone defect, and subsequently firing the shaped product under conditions of 1100° C. or more and 1200° C. or less for 5 minutes or more and 60 minutes or less.

### BRIEF DESCRIPTION OF DRAWINGS

FIG. 1(a) is a schematic view showing the crystal structure of β-TCP, and FIG. 1(b) is a schematic view showing columns constituting the crystal structure of β-TCP;

FIG. 2 is a schematic view showing a solid solution form of sodium ions in the column A constituting the crystal structure of β-TCP;

FIG. 3 is a schematic view showing the homogeneous structure of a single-phase bone prosthesis material according to the present invention;

FIG. 4 is a schematic view showing the heterogeneous structure of a composite bone prosthesis material according to the present invention;

FIG. 5 is a schematic scheme of a method for producing β-TCP and β-TCP containing monovalent cations in solid solution according to the present invention;

FIG. 6 is a schematic scheme of a method for manufacturing a bone prosthesis material sample composed of a β-TCP composite material containing monovalent cations in solid solution and a single-phase (3-TCP material containing monovalent cations in solid solution according to the present invention;

FIG. 7 is a view showing the results measured with an electron probe microanalyzer (EPMA) of a 4.6 mol % single-phase material manufactured by the manufacturing method according to the present invention;

FIG. 8 is a view showing the results measured with an EPMA of a 4.6 mol % composite material manufactured by the manufacturing method according to the present invention;

FIG. 9 is a graph showing the measurement results of solubility test on bone prosthesis material samples manufactured by the manufacturing method according to the present invention;

FIGS. 10(a) to 10(d) show electron micrographs after the solubility test of the bone prosthesis material samples manufactured by the manufacturing method according to the present invention, in which FIG. 10(a) and FIG. 10(b) show electron micrographs of the 4.6 mol % single-phase material and FIG. 10(c) and FIG. 10(d) show electron micrographs of the 4.6 mol % composite material;

FIG. 11 is a graph showing the results of an MTT assay using osteoclast-like cells on the bone prosthesis material samples manufactured by the manufacturing method according to the present invention; and

FIG. 12 is a graph showing TRAP activity levels, in an experiment using osteoclast-like cells, of the bone prosthesis material samples manufactured by the manufacturing method according to the present invention.

### DESCRIPTION OF EMBODIMENTS

In a process of sintering β-TCP, an abrupt volume contraction occurs in the range from 1000° C. to 1200° C. (the contraction rate is maximized at 1100° C.). The abrupt volume contraction leaves voids inside, which remain as closed pores and exert a detrimental effect on achieving a β-TCP closely packed material. It is considered that this is due to vacancies located in Ca positions in a β-TCP crystal structure (Non Patent Literatures 1 and 2). Monovalent cations such as sodium ions, which substitute and become solid solution also in β-TCP vacancies, have a high diffusion rate in crystals and additionally, grains grow larger in the sintering process (Non Patent Literatures 3 and 4). Accordingly, solid-solubilizing β-TCP and sodium ions in an amount within the solid solution limit by a conventional technique only provided a material having a homogeneous phase.

The inventors thus have made studies on a process for manufacturing a bone prosthesis material and on time and temperature of sintering for manufacturing a bone prosthesis material composed of a heterogeneous composite material and have succeeded in producing a heterogeneous composite material composed of a plurality of β-TCP phases containing monovalent cations in solid solution at different concentrations, more specifically, a composite material having a microdomain structure of a β-TCP phase containing monovalent cations in solid solution and a β-TCP phase. A bone prosthesis material according to one embodiment of the present invention will be described hereinbelow, referring to the drawings.

It should be noted that solid solution means that two or more elements are dissolved in each other to form a totally homogeneous solid phase. A sintered compact refers to a product solidified by heating at a temperature lower than its melting point.

### (1) Solid Solution Form

The β-TCP according to the present invention is formed by two phase components: β-TCP containing calcium ions (Ca²⁺) in crystals, wherein the calcium ions have been substituted with sodium ions (Na⁺) to be in solid solution, and β-TCP containing no solid solution component. The solubility of the β-TCP is influenced by its crystal structure, crystallinity (such as particle size). Accordingly, calcium ions in the crystals in a predetermined amount can be substituted with sodium ions to form a solid solution, which is then allowed to form a mixed phase with the β-TCP containing no solid solution component, thereby controlling the solubility of the resulting composite material.

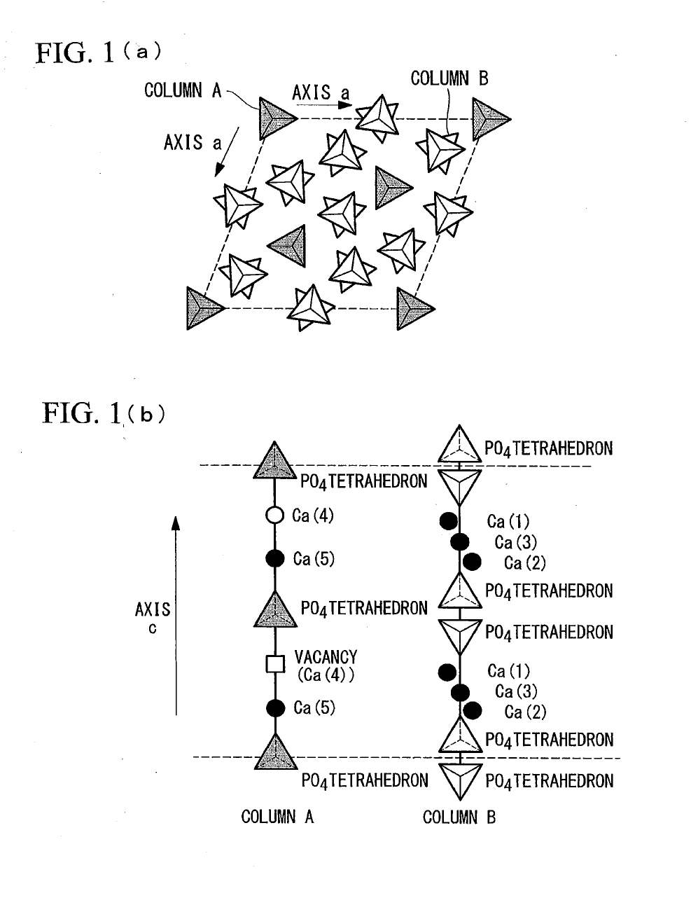

FIGS. 1A and 1B show the crystal structure of the β-TCP. As shown in FIG. 1(a), the space group of the β-TCP is R3c, which belongs to the rhombohedral system. Lattice constants are a=1.0439 nm and c=3.7375 nm in a hexagonal lattice setting. As shown in FIG. 1(b), the β-TCP has crystallographically-independent two columns A and B composed of Ca and PO₄ tetrahedrons in the crystal structure. The columns exist in parallel with the axis c. The column A includes repeats of PO₄(1)-Ca(4)-Ca(5)-PO₄(1)-□ (□: vacancy)-Ca(5). Since the Ca(4) site has a site occupancy of about 0.5, the column A is a crystal structure including a vacancy. The column B includes repeats of PO₄(3)-Ca(1)-Ca(2)-Ca(3)-PO₄(2)-PO₄(3)-Ca(1)-Ca(2)-Ca(3)-PO₄(2). The three Ca sites in the column do not fit on the c axis, forming a polygonal line. That is, a unit lattice of the β-TCP has crystallographically-independent three PO₄ sites and five Ca sites therein.

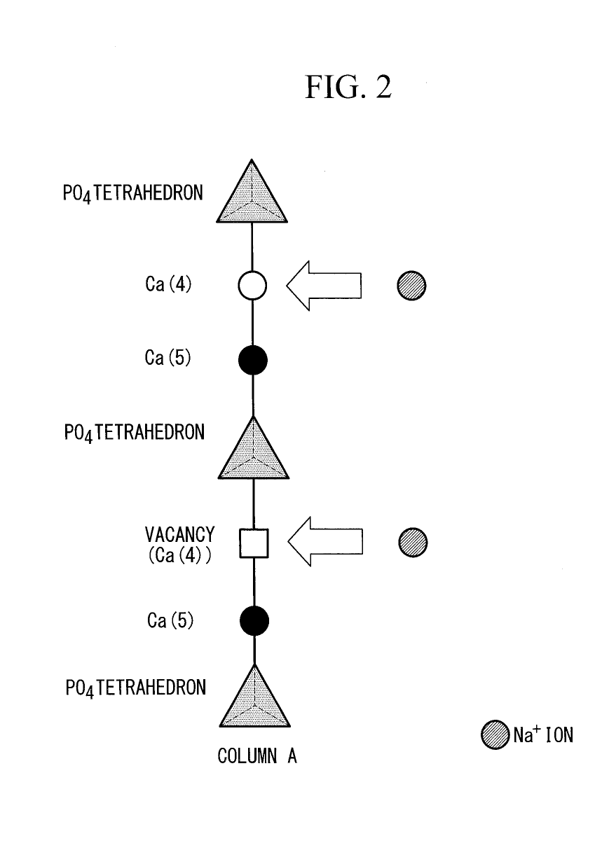

As shown in FIG. 2, monovalent ions (M⁺) such as sodium ions are in solid solution in the Ca(4) site and vacancy in the column A in the form of 2M⁺=Ca²⁺ ion+□ (□: vacancy), and their solid solution limit is known to be 9.1 mol %. It is also known that the degree of the solid solution of the cation in the β-TCP gives influences on the sinterability and solubility. Thus, in the present invention, β-TCP containing monovalent cations such as sodium ions in solid solution is prepared. A plurality of the β-TCP phases containing the monovalent cations in solid solution at different concentrations are used to form a composite material in which a plurality of phases containing monovalent cations in solid solution at different concentrations form a heterogeneously mixed phase (the schematic view of the composite material is shown in FIG. 4), providing a bone prosthesis material of which solubility can be controlled. It should be noted that the Ca(4) site of the column A in the β-TCP crystal structure is an atom position characteristic of taking a planar three-coordinate structure with oxide ions and having weak binding strength. Solid-solubilizing monovalent cations in this site provides a distorted six-coordinate structure, but the structure stability is not increased compared with other calcium sites. Accordingly, even if a difference in the solubility is caused by different solid solution concentrations, the difference in the solubilizing properties will not be large enough to cause problems such as weakening and reduced mechanical strength (Non Patent Literature 5).

### (2) Production of β-TCP and β-TCP Containing Monovalent Cations in Solid Solution

A β-TCP single phase containing no sodium ions in solid solution (0 mol), a β-TCP single phase containing 4.6 mol % of sodium ions in solid solution (4.6 mol single phase), and a β-TCP single phase containing 9.1 mol % of sodium ions in solid solution (9.1 mol), which are used for manufacturing a bone prosthesis material according to the present invention, can be produced according to an existing method. One example is shown in FIG. 4.

Diammonium hydrogen phosphate ((NH₄)₂HPO₄) as the phosphate source and calcium carbonate (CaCO₃) as the calcium source were used as the starting material, and Na₂CO₃ was used as the monovalent cation source. By use of a constant molar ratio of (Ca+Na+□)/P of 1.571 (a composition by taking □ (vacancy) in the structure into the account) and Na (mol %)=Na/(Ca+Na+□), a predetermined amount of monovalent cations in solid solution was blended (the amount of sodium ions was indicated as mol % relative to all the cation positions. Thus, an increase in the amount of the sodium ions leads to reduction in the amount of the calcium ions and □.). The starting material described above was wet-mixed in an organic solvent such as ethanol with an alumina ball mill for 48 hours. Subsequently, ethanol was removed using a rotary evaporator. The mixture after solvent removal was calcined at 900° C. for 12 hours in an ambient atmosphere. The resulting calcined mixture was dry-mixed in an agate mortar for an hour. The resulting mixture was calcined at 900° C. for 12 hours in an ambient atmosphere to thereby provide the intended β-TCP and β-TCP containing a predetermined amount of monovalent cations in solid solution.

### (3) Production of a Bone Prosthesis Material Sample Composed of a 4.6 Mol % Solid-Solution β-TCP Composite Material and a 4.6 Mol % Solid-Solution β-TCP Single-Phase Material

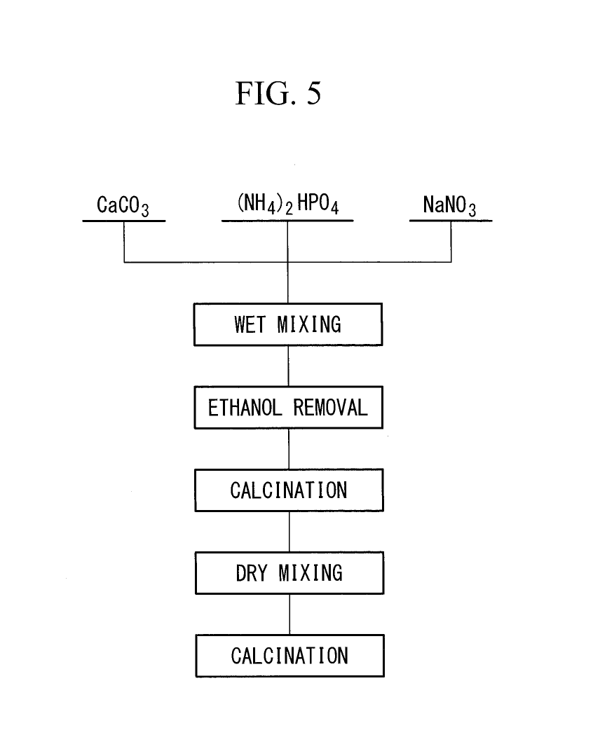

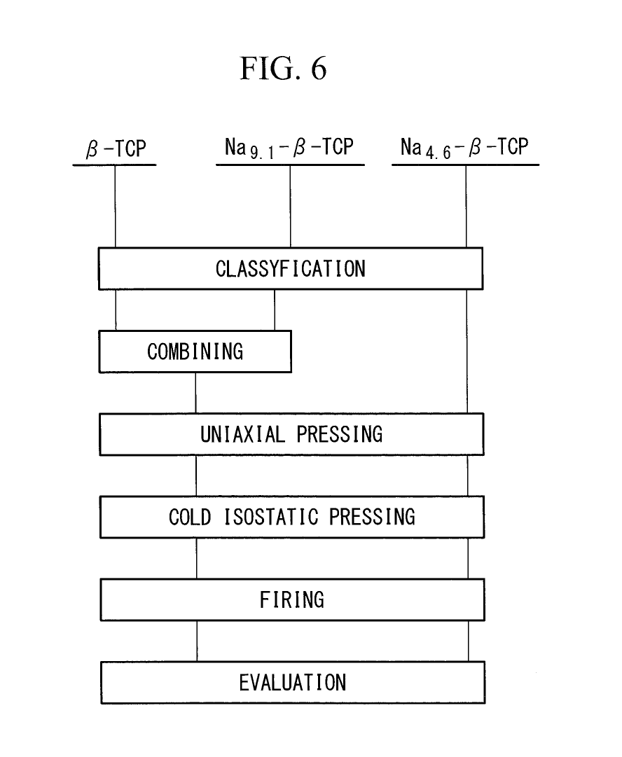

A β-TCP single-phase material containing 4.6 mol % of sodium ions in solid solution (4.6 mol single-phase material) and a β-TCP composite material containing 4.6 mol % of sodium ions in solid solution (composite material) can be produced according to an existing method. One example is shown in FIG. 5.

The β-TCP and the β-TCP containing monovalent cations in solid solution provided by the method described above were used as the starting material. A 1 wt % polyvinyl alcohol (PVA) aqueous solution was added to the starting material to aggregate the raw material powders, which were each classified with a sieve so as to have a particle size of 108 μm. In the case of forming the β-TCP composite material (composite material) containing 4.6 mol % of sodium ions in solid solution, a sample was provided by mixing (combining) classified samples of the β-TCP containing no sodium ions in solid solution and the β-TCP containing 9.1 mol % of sodium ions in solid solution in a molar ratio of 1:1 (mixing step). The resulting mixture and the 4.6 mol single-phase material were each subjected to uniaxial pressing at 32 MPa for one minute to form compacts, which were fired (firing step) to thereby provide sintered compacts. The mold used had a size of φ10 mm×3 mm or φ6 mm×1 mm. Firing was performed under conditions of a temperature rising rate of 3° C./min, a firing temperature of 1100° C. to 1200° C., and a retention time of 5 to 60 minutes in an ambient atmosphere.

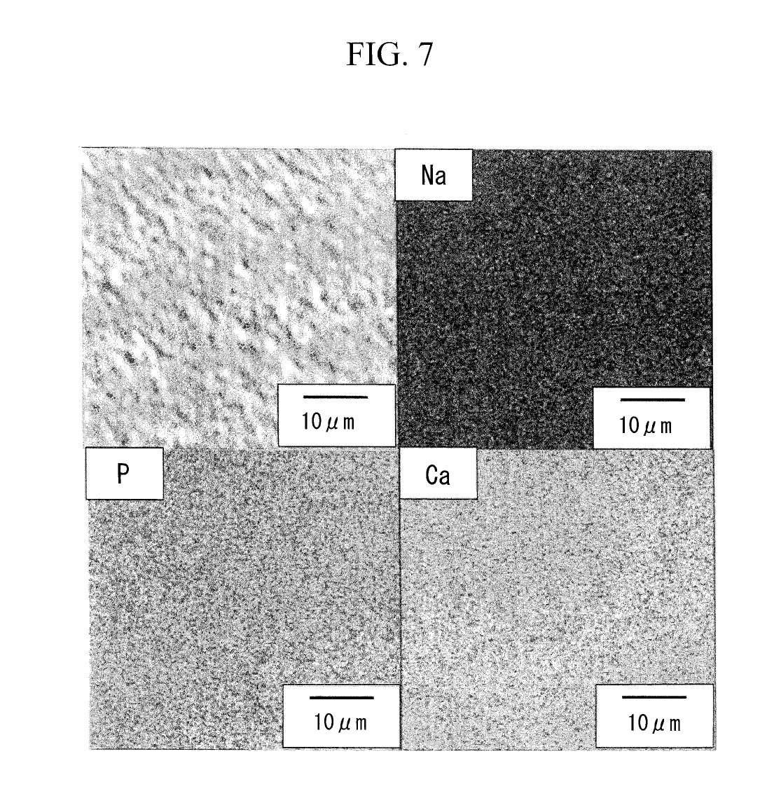

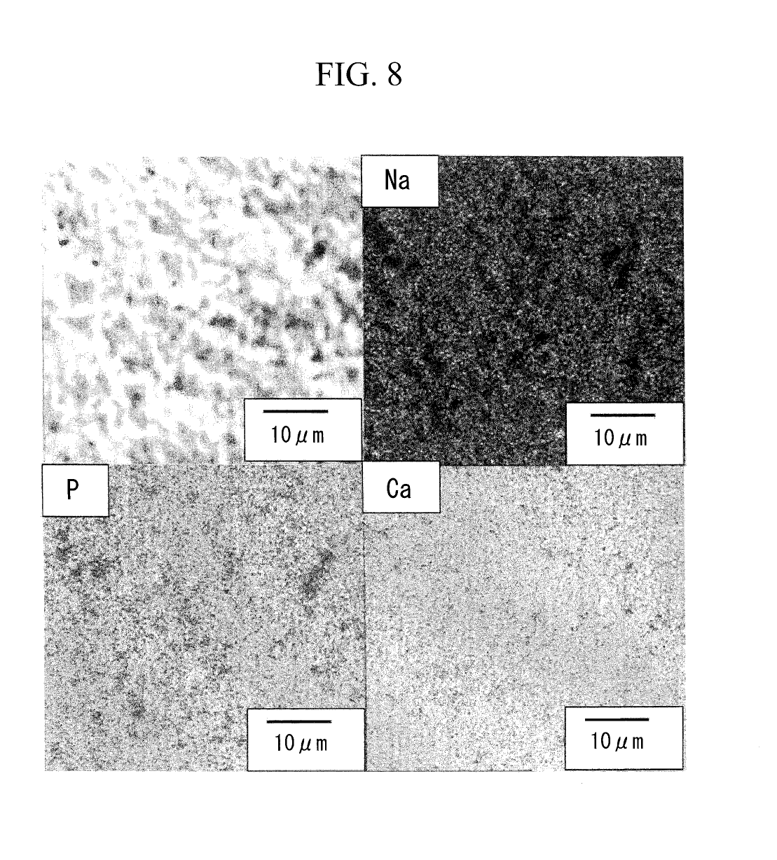

The electron-probe-microanalyzer images of the surface of the resulting 4.6 mol % solid-solution β-TCP single-phase material and 4.6 mol % solid-solution β-TCP composite material are shown in FIG. 7 and FIG. 8, respectively. In FIG. 7, which shows the single-phase material, Na, P, and Ca are spread homogeneously over each entire image. In contrast, in FIG. 8, which shows the composite material, portions where Na, P, and Ca are localized and portions where they are not localized are mixed in each image. This can confirm that the bone prosthesis material sample in which the β-TCP phase and the β-TCP phase containing monovalent sodium ions form a heterogeneously mixed phase can be provided by the manufacturing method according to the present invention.

Producing a sample composed of a single-phase material generally takes a sintering period of 24 hours or more. Meanwhile, monovalent cations such as sodium ions are known to have a high diffusion rate in crystals. Thus, when β-TCP and sodium ions were solid-solubilized using a conventional technique, sodium ions diffused in the solid solution to result in only a material composed of a homogeneous phase. In contrast, it has been able to be confirmed that firing a compact for a range of 5 to 60 minutes by the manufacturing method according to the present invention can provide the intended composite material, that is, a composite material having a microdomain structure in which a β-TCP phase containing monovalent cations in solid solution and a β-TCP phase form a heterogeneously mixed phase, without diffusing sodium ions.

Additionally, according to the present invention, in the process of classifying β-TCP and β-TCP containing monovalent cations in solid solution, which are the starting material, adjustment of the sieve opening size also can control the size of domains to be formed.

### (Solubility in Bioabsorbability Test In Vitro)

The bone prosthesis material samples provided by the aforementioned manufacturing method were subjected to bioabsorbability test in vitro.

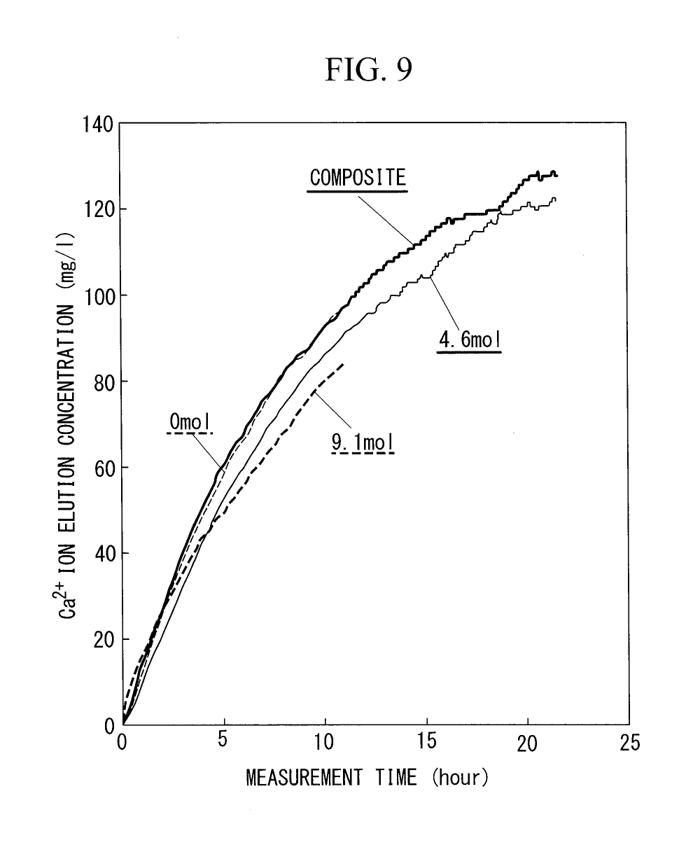

Each sample of a β-TCP single-phase material containing no sodium ion in solid solution (0 mol), a β-TCP single-phase material containing 4.6 mol % of sodium ions in solid solution (4.6 mol single phase), a β-TCP single-phase material containing 9.1 mol % of sodium ions in solid solution (9.1 mol), and a β-TCP composite material containing 4.6 mol % of sodium ions in solid solution (composite material) was hung with a nylon fishing line and immersed in an acetic acid-sodium acetate buffer solution of pH 5.50 at 25° C. under stirring for 0 to 24 hours to determine the amount of calcium ions eluted. The results are shown in FIG. 9. The horizontal axis represents the measurement time, and the vertical axis represents the elution concentration of the calcium ions in FIG. 9. In FIG. 9, “0 mol” signifies the β-TCP single-phase material containing no sodium ion in solid solution, “4.6 mol” signifies the β-TCP single-phase material containing 4.6 mol % of sodium ions in solid solution, “9.1 mol” signifies the β-TCP single-phase material containing 9.1 mol % of sodium ions in solid solution, and “Composite” signifies the β-TCP composite material containing 4.6 mol % of sodium ions in solid solution.

As shown in FIG. 9, it has been found that the solubility of the bone prosthesis material sample composed of the composite material according to the present invention was comparable to that of the non-solid-solution β-TCP at the early stage of the bioabsorbability test in vitro, specifically from the beginning of the measurement until 12 hours passed. That is, it has been able to be confirmed that the bone prosthesis material composed of the composite material according to the present invention has solubility sufficient to attract osteoclasts.

### (Confirmation of Composite Material (Domain Structure) Formation)

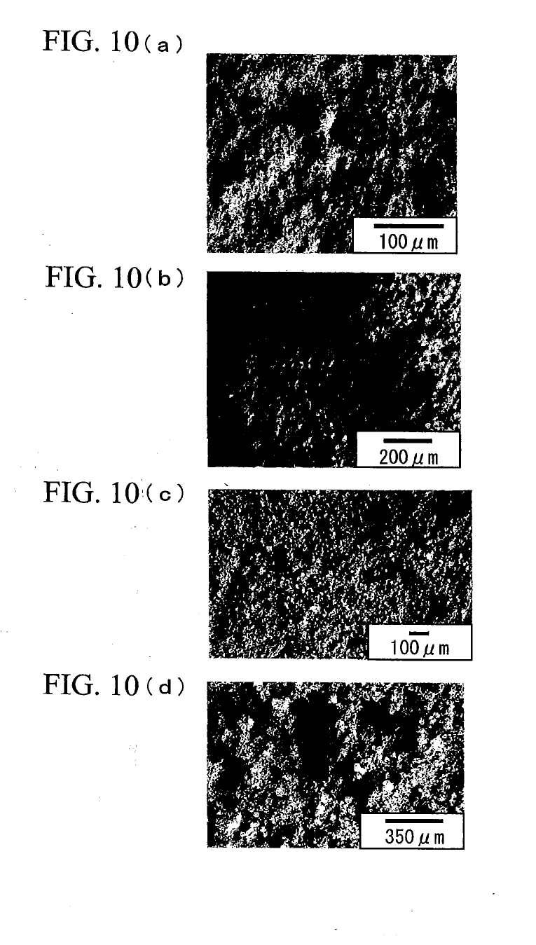

The scanning electron micrographs of the surface of the 4.6 mol single-phase material and composite material samples after the aforementioned bioabsorbability test are shown in FIGS. 10(a)-10(d). For the bone prosthesis material samples manufactured by the method according to the present invention, the single-phase material sample (FIGS. 10(a) and (b)) has irregularities spread substantially homogeneously on the sample surface, whereas the composite material sample (FIGS. 10(c) and (d)) has both significantly recessed portions and relatively flat portions having few irregularities. That is, this indicates that the bone prosthesis material sample manufactured by the method according to the present invention has voids formed in the portions where only the β-TCP phase dissolved under conditions of this experiment. Accordingly, it has been able to be confirmed that the bone prosthesis material manufactured by the method according to the present invention takes the form of a composite material in which the phases form a heterogeneously mixed phase (a domain structure).

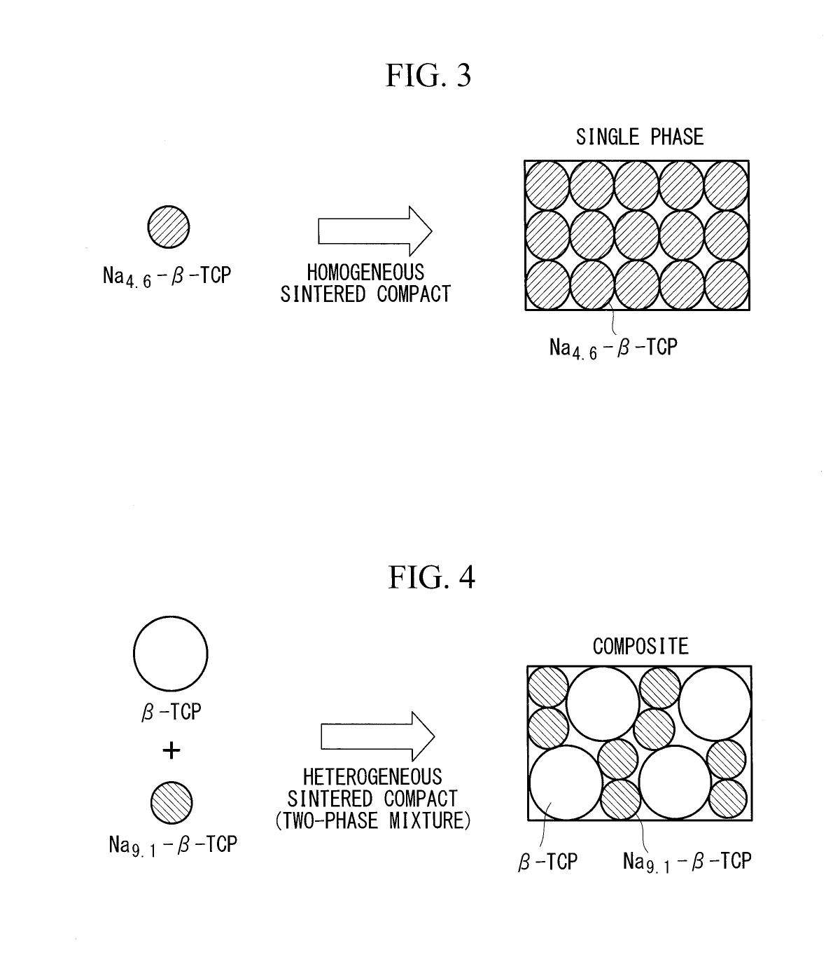

In the case of using a homogeneously mixed material (a single-phase material, the schematic view of which is shown in FIG. 3.), voids are unlikely to occur because dissolution proceeds homogeneously. In contrast, as in the present invention, in a heterogeneously mixed phase (composite material) composed of a highly-soluble β-TCP phase and a low-soluble β-TCP phase containing monovalent cations in solid solution, the highly-soluble β-TCP dissolves first, and voids occur in its dissolved portions. In such voids, it is possible to cause new osteogenesis as well as to allow remodeling to proceed.

### (Evaluation Using Osteoclasts-Like Cells)

Evaluation test using osteoclasts-like cells was performed on the samples provided by the aforementioned method, including a 4.6 mol % solid-solution β-TCP composite material (composite material) and a 4.6 mol % solid-solution β-TCP single-phase material (single-phase material). The composite material and the single-phase material used were formed by firing at 1150° C. for 10 minutes in accordance with the aforementioned method.

C7 cells, i.e., macrophage-like cells established from mouse bone marrow, were seeded on the composite material and the single-phase material each in a 48 well-plate at 5.0×10⁴ cells/ml, cultured for 3 hours, 1, 2, 3, and 7 days, and subsequently, subjected to evaluation by an MTT assay and TRAP stain. The culture medium used was 10% FBS-containing α-MEM supplemented with 1% Penicillin/Streptomycin and 0.5 ng/ml M-CSF. The differentiation-inducing factors used were 20 ng/ml RANKL 10⁻⁸ M 1α, 25(OH)₂D₃, and 10⁻⁷ M dexamethasone. The number of the samples subjected to the evaluation, n, is 10.

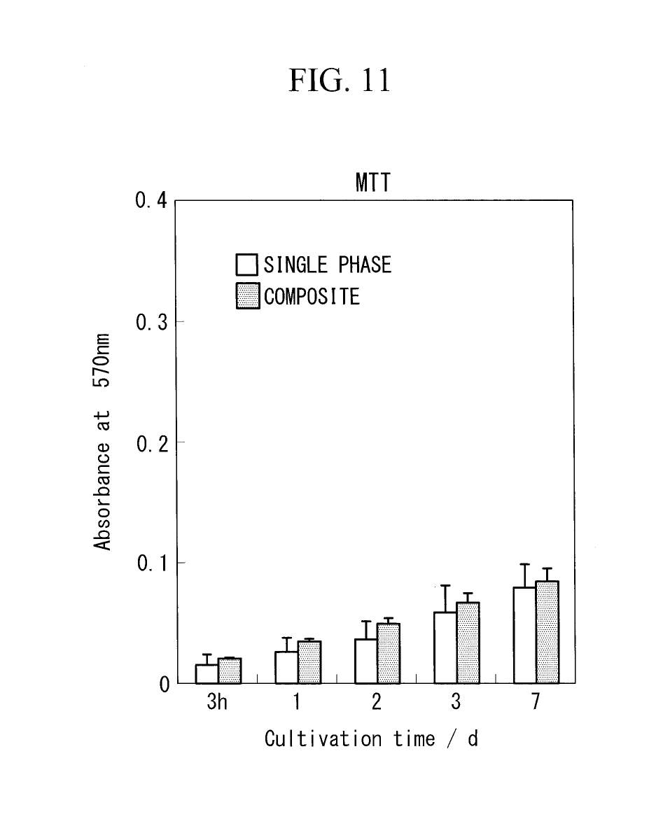

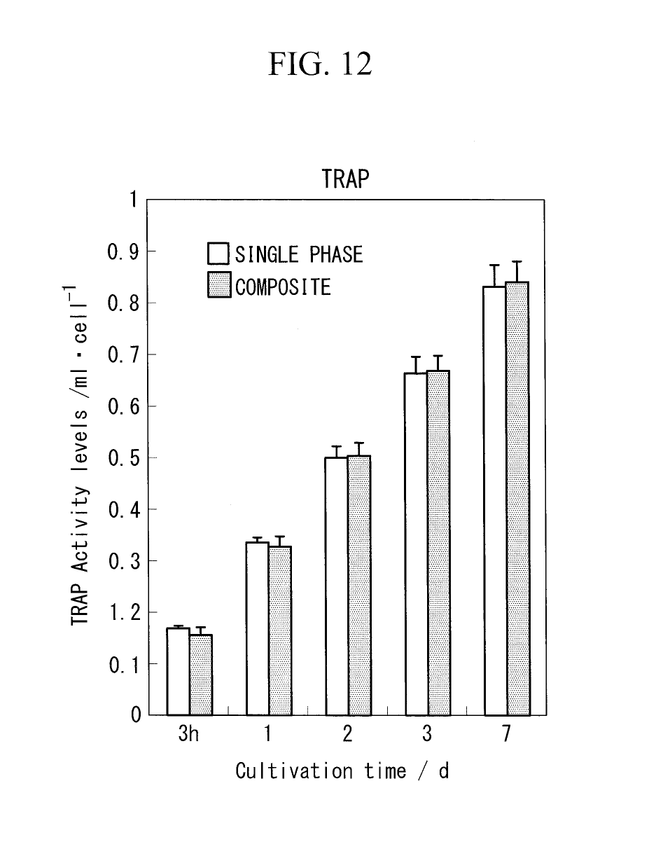

The result of the MTT assay is shown in FIG. 11, and the result of the TRAP stain is shown in FIG. 12. The osteoclast activities both on the composite material and on the single-phase material were almost equivalent. Additionally, in FIGS. 11 and 12, there was no statistically significant difference, at p<0.05, between the test data of the composite material and that of the single-phase material. That is, it has been found that the bone prosthesis material composed of the composite material according to the present invention retains the osteoclast activity comparable to that of the solid-solution single-phase material while retaining the solubility comparable to that of the non-solid-solution β-TCP.

From the above, the bone prosthesis material composed of the composite material provided by the present invention can promote elution of calcium ions while retaining the effect on osteoclasts. That is, the bone prosthesis material according to the present invention can attract osteoclasts due to its solubility comparable to that of β-TCP and enables osteoclasts to adhere thereto due to its osteoclast activity equivalent to that of β-TCP containing monovalent cations in solid solution. Accordingly, the bone prosthesis material according to the present invention enables its binding to a bone at an early stage.

The following aspects can be derived from the embodiment described above.

One aspect of the present invention provides a bone prosthesis material that is composed of a plurality of β-tricalcium phosphate (β-TCP) phases containing monovalent cations in solid solution at different concentrations.

According to the above aspect, being a composite phase of β-TCP phases containing monovalent cations in solid solution at different concentrations, the material promotes elution of calcium ion while retaining its effect on osteoclasts and enables its binding to a bone at an early stage.

In the bone prosthesis material according to the above-described aspect, the plurality of β-TCP phases containing monovalent cations in solid solution at different concentrations may form a heterogeneously mixed phase.

The bone prosthesis material according to the above-described aspect may include a β-TCP phase containing monovalent cations in solid solution and a non-solid-solution β-TCP phase.

Combination of a highly-soluble non-solid-solution β-TCP phase with a low-soluble β-TCP phase containing monovalent cations in solid solution allows osteoclasts to be attracted and to adhere.

That is, the β-TCP phase containing monovalent cations in solid solution dissolves gradually in a living body due to its controlled bioabsorbability compared with the non-solid-solution β-TCP phase having high bioabsorbability. Accordingly, bone metabolism in the portion of the non-solid-solution β-TCP phase is promoted whereas bone metabolism in the portion of the β-TCP phase containing monovalent cations in solid solution, which has controlled bioabsorbability, is enabled to gradually proceed.

In the above-described aspect, the monovalent cations may be sodium ions.

In this manner, a β-TCP phase containing monovalent cations in solid solution can be achieved, wherein the phase has an easily-controllable absorption rate in a living body as well as an excellent osteogenic ability, which is an important function for a bone prosthesis material.

The bone prosthesis material according to the above-described aspect may be formed by mixing particles of β-TCP containing monovalent cations in solid solution at different concentrations, shaping the mixture by a pressing method or a cutting shaping method into a shape suitable for a bone prosthesis material, that is, a cylinder, a rectangular parallelepiped, a polygonal column, a sphere, an ellipsoid, a granule, or a shape formed using three-dimensional data so as to precisely fit to a bone defect, and subsequently firing the shaped product under conditions of 1100° C. or more and 1200° C. or less for 5 minutes or more and 60 minutes or less.

This can achieve sintering without diffusing the monovalent cations in the crystals and form a composite material (microdomain structure) composed of a plurality of β-TCP phases containing monovalent cations in solid solution at different concentrations.

Another aspect of the present invention provides a method for manufacturing a bone prosthesis material comprising steps of: mixing particles of β-TCP containing monovalent cations in solid solution and particles of non-solid-solution β-TCP to provide a mixture; and shaping the mixture by a pressing method or a cutting shaping method into a shape suitable for a bone prosthesis material, that is, a cylinder, a rectangular parallelepiped, a polygonal column, a sphere, an ellipsoid, a granule, or a shape formed using three-dimensional data so as to precisely fit to a bone defect, and subsequently firing the shaped product under conditions of 1100° C. or more and 1200° C. or less for 5 minutes or more and 60 minutes or less.

The present invention can provide a bone prosthesis material that promotes elution of calcium ions while retaining its effect on osteoclasts. That is, the present invention can provide a bone prosthesis material that is a heterogeneous composite material (having a microdomain structure) composed of a highly-soluble phase to attract osteoclasts and a low-soluble phase to enable the osteoclasts to adhere thereto, achieving the effect of binding the material to a bone at an early stage

1. A bone prosthesis material comprising a plurality of β-tricalcium phosphate (β-TCP) phases containing monovalent cations in solid solution at different concentrations.

2. The bone prosthesis material according to claim 1, wherein the plurality of β-TCP phases containing monovalent cations in solid solution at different concentrations form a mixed heterogeneously mixed phase.

3. The bone prosthesis material according to claim 1 comprising a β-TCP phase containing monovalent cations in solid solutions and a non-solid-solution β-TCP phase.

4. The bone prosthesis material according to claim 1, wherein the monovalent cations are sodium ions.

5. The bone prosthesis material according to claim 1, wherein the material is formed by mixing particles of the β-TCP containing monovalent cations in solid solution at different concentrations, shaping the mixture into a predetermined shape, and subsequently firing the shaped product under conditions of 1100° C. or more and 1200° C. or less for 5 minutes or more and 60 minutes or less.

6. The bone prosthesis material according to claim 5, wherein the predetermined shape is a cylinder, a rectangular parallelepiped, a polygonal column, a sphere, an ellipsoid, a granule, or a shape formed using three-dimensional data so as to precisely fit to a bone defect.

7. A method for manufacturing a bone prosthesis material comprising steps of:
mixing particles of β-TCP containing monovalent cations in solid solution at different concentrations to provide a mixture; and
shaping the mixture into a predetermined shape, and subsequently firing the shaped product under conditions of 1100° C. or more and 1200° C. or less for 5 minutes or more and 60 minutes or less.

8. The method for manufacturing a bone prosthesis material according to claim 7, wherein the predetermined shape is a cylinder, a rectangular parallelepiped, a polygonal column, a sphere, an ellipsoid, a granule, or a shape formed using three-dimensional data so as to precisely fit to a bone defect.

9. The method for manufacturing a bone prosthesis material according to claim 7, wherein the predetermined shape is formed by a pressing method or a cutting shaping method, or a combination thereof.
